(19) United States
(12) Patent Application Publication
(10) Pub. No.: US 2015/0010501 A1
(43) Pub. Date: Jan. 8, 2015

(54) INTERVERTEBRAL DISC REPAIR COMPOSITIONS AND METHODS

(71) Applicant: Parcell Laboratories, LLC, Natick, MA (US)

(72) Inventors: Keith D. Crawford, Westwood, MA (US); Pamela Layton, Westwood, MA (US); John Garvey, Londonderry, NH (US); Jeffrey A. Gadboys, Gulf Breeze, FL (US)

(21) Appl. No.: 14/497,690

(22) Filed: Sep. 26, 2014

Related U.S. Application Data
(63) Continuation of application No. PCT/US13/32255, filed on Mar. 15, 2013.
(60) Provisional application No. 61/615,985, filed on Mar. 27, 2012.

(51) Int. Cl. A61K 35/24; A61K 35/14; C12N 5/074

(52) U.S. Cl. CPC A61K 35/24; C12N 5/0607; A61K 35/14; C12N 2501/105; C12N 2501/135; C12N 2501/11; C12N 2501/33; C12N 2501/998; C12N 2501/999
USPC 424/85.2; 435/325; 424/93.7; 435/283.1

(57) ABSTRACT

The present invention provides compositions, methods and devices for treatment of degenerative disc disease or the repair of a disc in need of repair. The composition or the formulation includes freshly isolated or culture expanded ELA cells, which can be cyropreserved. The composition or a formulation also includes in various embodiments an ELA cell population that has been differentiated into cell types having at least one characteristic of human intervertebral disc cells, such as fibroblast cells, chondrocyte cells or notochordal cells. The composition or the formulation includes in certain embodiments either a population of ELA cells provided in conjunction with one or more biocompatible molecules, therapeutic agents, or agents that induce ELA stem cell differentiation. The ELA cells are obtained from fluid or tissue from live or cadaveric (cadaverous) donors. Treatment of a degenerative disc or a disc in need of repair includes in various embodiments one or more injections or implantations of the composition comprising the ELA cells within or in association with the disc in need of repair.

### RELATED APPLICATION

This international application claims the benefit of U.S. provisional application Ser. No. 61/615,985 filed Mar. 27, 2012 in the U.S. Patent and Trademark Office, entitled, “Intervertebral disc repair compositions and methods” by Keith D. Crawford, Pamela Layton, John Garvey, and Jeffrey Gadboys, the entirety of which is hereby incorporated herein by reference in its entirety.

### FIELD OF THE INVENTION

The present invention relates to compositions, methods and kits using adult stem cells for the treatment of back pain, inflammation, and degenerative disc disease.

### BACKGROUND

Intervertebral discs absorb and dissipate the compressive force applied to the spine by gravity. The discs allow motion between vertebral bodies in planes of motion. Deformability of the intervertebral discs allow for the distribution of forces over the entire surface area of the vertebral body endplate rather than focusing loading and torsional forces at the periphery of the vertebral body (Connell, M. D. et al. 1992 Ortho Clin North Am, Vol. 23, No. 3; and Fisher, D. K. et al., 1992 “Spinal spondylosis and disc disease”, in Evans R. W., Baskin, D. S., and Yatsu, F. M., Prognosis of Neurological Disorders, Oxford University Press, New York, 1992.). The gelatinous nucleus pulposus acts as a hydrodynamic ball bearing that converts the vertical pressures of axial loading to horizontal forces that can be absorbed by the annulus fibrosus The natural elasticity of the fibrous rings allows an increase in the diameter of the discs. This small amount of movement in the horizontal plane helps in the spine's stability.

Degenerative changes in the spine and disc cause the loss of normal structure and function. The intervertebral discs are prone to the degenerative changes associated with wear and tear aging, even misuse (e.g. smoking). This degeneration leads to a reduction in the thickness and compressibility of the disc and can lead to very severe sensations of pain.

When degeneration reaches an advanced stage, it is often necessary to remove one or more natural intervertebrals disc and to replace them. If an entire disc is removed, spinal column instability may warrant fusion. Spinal fusion, the conventional treatment for a degenerative disc, is often combined with spinal instrumentation, the use of medically designed hardware, e.g. screws, cages. Fusion involves placing bone graft from the patient's pelvic bone and inserting metal rods or cages to stabilize the spine. Although spinal fusion can relieve pain by eliminating movement at the motion segment, it often decreases the patient's functional range of motion and may increase stress to the adjacent discs and facet joints. Spinal fusion also presents the drawback, particularly if applied to several vertebrae, of considerably limiting the patient's ability to move. Alternatively, the disc may be completely replaced with an artificial disc, which is mounted between the vertebrae and which, ideally, conserves for the patient all of the relative mobility between the vertebrae, or at least a large fraction thereof

Thus, there remains a need for effective therapies to repair damage to intervertebral disc and in spinal tissues, and to reduce pain and inflammation for patients receiving these therapies.

### SUMMARY

An aspect of the invention provides a composition including: a preparation of ELA adult stem cells. In various embodiments, the ELA stem cells are characterized by one or more stem cell-specific transcription factors, and lacking detectible expression of one or more of the cell surface markers: CD13, CD34, CD44, CD45, CD90, or MHC class I. In various embodiments, the ELA stem cells are characterized by expression of one or more of the stem cell specific genes: Oct-4, KFL-4, Nanog, Sox-2, Rex-1, GDF-3 and Stella, and lacking detectible expression of one or more of the cell surface markers: CD13, CD34, CD44, CD45, CD49a, CD66A, CD73, CD90, CD105, CSCR4, SSEA, MHC class I or MHC class II.

The terms “formulation”, “composition” and “pharmaceutical composition” are used interchangeably herein collectively refer to a plurality of ELA stem cells formulated or compounded with at least one other substance (e.g., excipient), agent, medium, or liquid.

In various embodiments, the composition is effective for treating a disc disease or condition. For example the disease or condition is scoliosis, degenerative disc disease, kyphosis, cervical spondylosis, failed back surgery syndrome (FBSS), herniated nucleus pulposus, slipped disc, lumbar radiculopathy, cervical radiculopathy, herniated intervertebral disc, prolapsed intervertebral disc, ruptured disc, spinal surgery, disc surgery, or ankylosing spondylitis.

In certain embodiments, the composition further includes a pharmaceutically acceptable excipient. In various embodiments, the composition including the ELA stem cells is effective for treating a spinal or disc condition, e.g., a disc injury. For example, the composition is effective for treating a disc herniation or a degeneration of an intervertebral disc. In various embodiments, the composition is effective for treating a joint. In various embodiments, the composition is effective for treating a knee, a shoulder, a hip, an ankle, or a wrist. In various embodiments, the composition is effective for reducing at least one selected from: pain, discomfort, and inflammation.

In certain embodiments, the composition further includes a contrast agent. For example, the contrast agent is a perfluorate agent for imaging a spine, a disc, or an adjacent tissue (e.g., muscle and nerve). For example, the contrast agent is an ultrasound contrast agent.

In certain embodiments, the composition further includes an analgesic agent, e.g., a drug and compound. For example, the analgesic agent includes a non-steroidal anti-inflammatory drug (NSAID) such as a salicylate, an opioid drug such as morphine, or a derivative or portion thereof. In certain embodiments, the analgesic agent includes a COX-2 inhibitor, or a derivative or portion thereof. In various embodiments, the analgesic agent is further an inhibitor of inflammation.

In various embodiments, the composition further includes an anti-pyretic or an anti-inflammatory.

The composition in certain embodiments further includes a radiographic agent. In certain embodiments the radiographic agent includes a high-osmolar contrast medium, a low-osmolar contrast medium, or an iso-osmolar contrast medium. For example, the radiographic agent includes a diatrizoate, an iohexol, or an iodixanol.

In various embodiments the composition further includes a biocompatible matrix. In certain embodiments of the composition, the matrix is at least one selected from the group of: a gel, a tissue graft, a polymer, a scaffold, calcium triphosphate, demineralized bone, collagen, and cellulose. For example, the tissue graft includes a bone graft. In various embodiments, the matrix conforms substantially to a site of insertion in the subject and provides a structurally stable three-dimensional surface. In certain embodiments, the matrix supports ingrowth of the ELA stem cells at the site of the insertion. The matrix in various embodiments of the composition promotes ingrowth of the ELA stem cells and induces tissue formation, for example the tissue growth includes a type of tissue selected from ectoderm, mesoderm, and endoderm. In certain embodiments, the ELA stems growing in or through the matrix result in the formation of at least one type of tissue selected from the group of: connective, bone, dermal, neuronal, endothelial tissue, cartilage, and muscle. In various embodiments, the biocompatible matrix is a naturally-occurring material or a synthetic material. In various embodiments, the biocompatible matrix includes an extracellular matrix for tissue formation or growth.

The composition in various embodiments is administered prior to, contemporaneously, or subsequent to the biocompatible matrix being inserted into subject. For example the ELA stem cells are administered after the biocompatible matrix is inserted at a site of a spine or disc disorder or condition. For example, the composition is administered minutes, hours, days or months after the biocompatible matrix is inserted. Alternatively, the composition is administered minutes, hours, days or months before the biocompatible matrix is inserted.

In various embodiments, the biocompatible matrix is obtained from an allogeneic donor or is obtained from an autologous source, i.e., from the same subject receiving the composition. In various embodiments, the biocompatible matrix is a graft material obtained from the subject or obtained from a blood relative of the subject, e.g., brother, sister, aunt, uncle, cousin, niece, nephew, and grandparent.

In various embodiments, the ELA stem cells are substantially free of erythrocyte cells. In a related embodiment, substantially free is about 90% free of erythrocyte cells, about 95% free of erythrocyte cells, or about 99% free of erythrocyte cells compared to a control set of stem cells that are not ELA stem cells.

In related embodiments, the composition further includes at least one selected from the group of: a serum, a plasma, a buffer, cell culture medium, a preservative, an anti-bacterial agent, an anti-fungal agent, a conditioning agent, a cryogenic agent, a pharmaceutically acceptable salt, a growth factor, a vitamin, a hormone, and a therapeutic agent. For example, the growth factor is a bone-morphogenic protein. In various embodiments, the therapeutic agent modulates expression of a gene or gene product in ELA stem cells. For example the gene product is a protein or a peptide. In various embodiments, the protein or the peptide is associated with the annulus or nucleus pulposus. In various embodiments, the protein or the peptide is associated with disc or bone formation.

In related embodiments, the therapeutic agent includes at least one selected from: an inorganic compound, a drug, a nucleic acid, a lipid, a peptide, a protein for example an antibody, and a carbohydrate. In related embodiments, the nucleic acid includes a DNA or an RNA. In related embodiments, the DNA includes cDNA. In related embodiments, the RNA includes at least one selected from: mRNA, tRNA, rRNA, siRNA, RNAi, miRNA, and dsRNA. In related embodiments of the method, the therapeutic agent includes a vector or a plasmid. In various embodiments, the vector carries a nucleotide sequence that encodes a protein. For example the protein is a growth factor. In various embodiments, the therapeutic agent is at least one selected from: a fibroblast growth factor (FGF) for example FGF2; an endothelial growth factor (EGF); a stem cell factor (SCF); an interleukin (IL) for example IL-6 or IL-2; a transforming growth factor (TGF) for example TGF-α or TGF-β, or a combination there of. In various embodiments, the therapeutic agent is a human peptide or a human protein.

In various embodiments, the therapeutic agent is osteogenic or neurogenic.

In related embodiments, the composition includes a unit dose of the ELA stem cells. For example the unit dose of the ELA stem cells is selected from: about 10² cells to about 10³ cells, about 10³ cells to about 10⁴ cells, about 10⁴ cells to about 10⁵ cells, about 10⁵ cells to about 10⁶ cells bout 10⁶ cells to about 10⁷ cells; 10⁷ to about 10⁸ cells; about 10⁸ cells to about 10⁹ cells; about 10⁹ cells to about 10¹⁰ cells; and about 10¹⁰ cells to about 10¹¹ cells.

In related embodiments, the ELA stem cells include cryogenically frozen cells, for example the frozen cells are from a stem cell bank. In various embodiments, the ELA stem cell bank is preserved or maintained at about −80° C., about −100° C., about −110° C., or about −120° C. In various embodiments, the ELA stem cells are preserved at a temperature below about −130° C. or below about −150° C. For example, the temperature is below a range of about −130° C. to about −140° C., about −140° C. to about −150° C., about −150° C. to about −160° C., about −160° C. to about −170° C., about −170° C. to about −180° C., and below a range of about −180° C. to below about −190° C. For example, the ELA stem cells are preserved at about −196° C. In various embodiments, the ELA stem cells are preserved using dry ice or liquid nitrogen. Alternatively, the ELA stem cells are freshly prepared. For example the ELA stem cells are prepared or cultured minutes or hours prior to preparing the composition or the preparation.

In various embodiments, the preparation of ELA stem cells is culture expanded. In various embodiments of the composition, the cultured cells are differentiated cells. For example, the differentiated cells are an osteogenic lineage for forming bone or a chondrogenic lineage for forming cartilage. In various embodiments, the ELA stem cells have a median size of about from about 3 micrometers (microns; μm) to 25 micrometers. For example, the ELA stem cells are a freshly isolated population. In various embodiments, the ELA stem cells have a size of about 3 micrometers to about 3.5 micrometers; about 3.5 micrometers to about 4 micrometers; about 4 micrometers to about 4.5 micrometers; about 4 micrometers to about 5 micrometers; about 5 micrometers to about 5.5 micrometers; and about 5.5 micrometers to about 6 micrometers, viz., about 3 micrometers to about 6 micrometers. In various embodiments, the ELA stem cells have a size of about 3 micrometers to about 5 micrometers, about 5 micrometers to about 7 micrometers, about 7 micrometers to about 9 micrometers, about 9 micrometers to about 11 micrometers, about 11 micrometers to about 13 micrometers, about 13 micrometers to about 15 micrometers, about 15 micrometers to about 17 micrometers, about 17 micrometers to about 19 micrometers, or about 19 micrometers to about 21 micrometers. For example, the ELA stem cells have a size of at least about 3 micrometers, at least about 5 micrometers, at least about 7 micrometers, at least about 9 micrometers, at least about 11 micrometers, at least about 13 micrometers, at least about 15 micrometers, at least about 17 micrometers, or at least about 19 micrometers.

In various embodiments, the formulation or the preparation of ELA stem cells further includes a nanostructure. In various embodiments the formulation or the preparation of ELA stem cells further includes at least one selected from the group of: a scaffold such as a collagen scaffold; a matrix; a nanocomposite for example which contains collagen-like or nucleus pulposus-like fibers, a microfiber, a nanofiber, or a nanotubule. For example, the matrix is a biocompatible matrix. In various embodiments, the scaffold is a self-assembly scaffold (also referred to as a temperature-dependent scaffold) that changes at least one of shape, viscosity, or size depending on the temperature. In various embodiments, the self-assembly scaffold includes a matrix or biocompatible material. In various embodiments, the self-assembly scaffold are liquid at room temperature and upon injection (for example with an 18 gauge hypodermic needle) into the body or body cavities becomes solid or semi-solid. For example, the self-assembly scaffold has a collagen-like form.

In various embodiments, the self-assembly scaffold is liquid at about room temperature and solidifies or re-assembles into a solid or semi-solid or gel at about body temperature. In various embodiments, the self-assembly scaffold solidifies or re-assembles at about 37° C.

In various embodiments, the nanostructure, the scaffold, the matrix, the nanocomposite, the microfiber, the nanofiber, or the nanotubule includes at least one of: the therapeutic agent, an inorganic compound, a drug, a nucleic acid, a lipid, a protein, a peptide, an antibody, and a carbohydrate. For example, the therapeutic agent, the inorganic compound, the drug, the nucleic acid, the lipid, the protein, the peptide, the antibody, or the carbohydrate enhance differentiation of the ELA stem cells. For example, the differentiation includes osteogenic differentiation or neurogeneic differentiation.

In various embodiments, the nanostructure, the scaffold, the matrix, the nanocomposite, the microfiber, the nanofiber, or the nanotubule includes a helical structure. For example, the helical structure allows the ELA stem cells to attach or lie on top of the nanostructure, the scaffold, the matrix, the nanocomposite, the microfiber, the nanofiber, or the nanotubule. In various embodiments, the helical structure enhances growth or function of the ELA stem cells.

In various embodiments of the formulation, the nanostructure, the scaffold, the matrix, the nanocomposite, the microfiber, the nanofiber, or the nanotubule is composed of at least one polymer. In various embodiments of the formulation, the polymer is further characterized by at least one property selected from the group of crystalline, amorphous, bio-resorbable, porous, elastic, and sterile. For example, porosity of the polymer enhances the controllable release of the therapeutic agent, or seeding or in-growth of the ELA stem cells. In a related embodiment, the polymer is a pure polymer having a distinct specific set of characteristics and properties, for example the polymer is entirely a poly-L-lactic acid or a poly-lactic-co-glycolide, or the polymer is entirely hydrophilic or hydrophobic. Alternatively, the polymer is a composite/blend material of at least two different materials (e.g., polymer, metal, nanodot composition). In a related embodiment, the nanostructure, the scaffold, the matrix, the nanocomposite, the microfiber, the nanofiber, or the nanotubule having the ELA stem cells is composed of interlocked/bound polymers contains different types of pure polymers or composite polymers, i.e., at least two polymers have different characteristics and properties.

In a related embodiment, the polymer further includes an additional agent that modulates strength or elasticity of the polymer, or that modulates release of the therapeutic agent from the polymer or that modulates seeding or ingrowth of the ELA stem cells. For example, the additional agent is at least one selected from: a polymer, an elasticizer, an emollient, a hardener, a carbon (e.g., a diamond, a graphite), a hydrocarbon, a nano-based composition, a composite material comprising at least two different types of substances, and the like. A nano-based composition for example includes: a nano-metal, a nano-ceramic, a nano-polymer, and the like.

An aspect of the invention provides a kit for treating a condition or disorder of a spine or disc of a subject, the kit including: a preparation of ELA adult stem cells; and a container. In various embodiments, the ELA stem cells are characterized by one or more stem cell-specific transcription factors, and lacking detectible expression of one or more of the cell surface markers: CD13, CD34, CD44, CD45, CD90, or MHC class 1. In various embodiments, the ELA stem cells are characterized by expression of one or more of the stem cell specific genes: Oct-4, KFL-4, Nanog, Sox-2, Rex-1, GDF-3 and Stella, and lacking detectible expression of one or more of the cell surface markers: CD13, CD34, CD44, CD45, CD49a, CD66A, CD73, CD90, CD105, CSCR4, SSEA, MHC class I or MHC class II.

In certain embodiments, the kit further includes a pharmaceutically acceptable excipient. In various embodiments, the preparation including the ELA stem cells is effective for treating a disc herniation or degenerative disc. In various embodiments, the kit further includes instructions for use. For example, the instructions include a method for treating a disc injury in a mammal using ELA stem cells, or a method of preparing the preparation containing the ELA stem cells and of administering the composition to the mammal or a subject.

In certain embodiments, the kit further includes a perfluorate agent. For example, the perfluorate agent is a contrast agent for imaging a spine, a disc, or adjacent tissues (e.g., muscle). For example, the contrast agent is an ultrasound contrast agent.

In certain embodiments, the kit further includes an analgesic agent, e.g., a drug or compound. For example, the analgesic agent includes a NSAID, an opioid drug, or a derivative or portion thereof. In certain embodiments, the analgesic agent includes a COX-2 inhibitor, or a derivative or portion thereof.

The kit in certain embodiments further includes a radiographic agent. In certain embodiments the radiographic agent includes a high-osmolar contrast media, a ow-osmolar contrast medium, or an iso-osmolar contrast medium. For example, the radiographic agent includes a diatrizoate, an iohexol, or an iodixanol.

The kit in various embodiments further includes a bioactive agent or a therapeutic agent. In various embodiments, the therapeutic agent modulates expression of a gene or gene product in ELA stem cells. In various embodiments, the bioactive agent or the therapeutic agent is at least one selected from a drug, a compound, a protein, a peptide, a carbohydrate, and a genetic material. For example, the genetic material is DNA or RNA. In various embodiments, the bioactive agent or the therapeutic agent is at least from the group consisting of: a growth factor, a cytokine, a chemokine, a bone morphogeneic protein, an IL-3 receptor agonist, an interleukin, and a transcription factor. In various embodiments, the bioactive agent or the therapeutic agent is a modulator of stem cell differentiation. For example, the modulator induces osteogenic differentiation.

In various embodiments, the kit further includes a biocompatible material or a biocompatible matrix. In related embodiments, the biocompatible material or the biocompatible matrix includes at least one of: 2-hydroxyethyl methacrylate, 2-hydroxypropyl methacrylate, demineralized bone, a cancellous bone, an allograft, an autograft, a hydrogel, gelatin, collagen, cellulose, bone graft synthetic substitute, and a combination thereof. In various embodiments, the biocompatible material or the biocompatible matrix includes at least one selected from the group consisting of: a gel, a graft, a scaffold, calcium triphosphate, demineralized bone, collagen, and cellulose. In various embodiments, the scaffold includes a polymer scaffold or a self-assembly scaffold. For example, the graft is a tissue graft, e.g., a bone graft or disc graft. In various embodiments, the biocompatible material or the biocompatible matrix includes a device, for example a stabilization device for a portion of the spine. In various embodiments, the stabilization device is an intervertebral stabilization device. In various embodiments, the stabilization device is a load-bearing device.

The kit in various embodiments further includes an applicator for administering the composition to the spine or the disc of a subject. For example, for example the applicator is at least one selected from the group of: a bottle, a sprayer, a dropper, a gauze, a strip, a brush, a syringe, and a catheter. In various embodiments, the kit further includes a vessel or a reservoir for culturing the ELA stem cells.

In various embodiments, the kit includes any of the compositions or formulations described herein.

An aspect of the invention provides a method for treating disc injury in a mammal, the method including: identifying a mammal having discogenic pain incident to an annular lesion or a degenerative disc; administering an effective amount of a composition including ELA stem cells within or in association with (e.g., contacting the external surface of) the annulus fibrosus (annulus) or the degenerative disc, thereby treating the annular lesion or degenerative disc, and thereby ameliorating or reducing pain in the mammal. In various embodiments, the mammal is a human subject. In various embodiments, the ELA stem cells are characterized by one or more stem cell-specific transcription factors, and lacking detectible expression of one or more of the cell surface markers: CD13, CD34, CD44, CD45, CD90, or MHC class I. In various embodiments, the ELA stem cells are characterized by expression of one or more of the stem cell specific genes: Oct-4, KFL-4, Nanog, Sox-2, Rex-1, GDF-3 and Stella, and lacking detectible expression of one or more of the cell surface markers: CD13, CD34, CD44, CD45, CD49a, CD66A, CD73, CD90, CD105, CSCR4, SSEA, MHC class I or MHC class II. In various embodiments, the administering the compositions involves using any of the compositions or the formulation described herein.

In various embodiments of the method, administering involves contacting adjacent tissues with the composition and allowing migration or diffusion of the ELA stem cells to the annulus or the degenerative disc. In various embodiments, administering involves topical administration to the degenerative disc.

In related embodiments of the method, administering includes applying the composition by injection, for example intraspinally, spinally, epidurally, intravenously, or intra-muscularly. In various embodiments, administering the composition involves directing the composition to the spinous process or an adjacent area of the spine or disc. In various embodiments, administering involves contacting the composition to a spinal endplate, an epidural space, or a spinal cord cavity. In various embodiments, administering involves using a cannula, a needle, a syringe, or an endoscope.

In various embodiments of the method, an effective amount of the composition comprises a population of ELA cells at a cell density between: about 10² cells per milliliter and about 10³ cells per milliliter, about 10³ cells per milliliter and about 10⁴ cells per milliliter, about 10⁴ cells per milliliter and about 10⁵ cells per milliliter, about 10⁵ cells per milliliter and about 10⁶ cells per milliliter, about 10⁶ cells per milliliter and about 10⁷ cells per milliliter, and about 10⁷ cells per milliliter and about 10⁸ cells per milliliter. For example, the effective amount of the composition includes about 10⁴ cells per milliliter and about 10⁷ cells per milliliter. In various embodiments, the cell density is about 10⁶ cells per milliliter and about 10¹² cells per milliliter.

In various embodiments of the method, the effective amount of the composition is in a volume of between about 0.25 milliliters (250 microliters) and about 10 milliliters. For example, the volume is about 0.25 milliliters (ml) to about 0.5 ml, about 0.5 ml to about 1 ml, about 1 ml to about 1.5 ml, about 1.5 ml to about 2 ml, about 2 ml to about 2.5 ml, about 2.5 ml to about 3 ml, about 3 ml to about 3.5 ml, about 3.5 ml to about 4.0 ml, about 4.0 ml to about 4.5 ml, about 4.5 ml to about 5 ml, about 5 ml to about 6 ml, about 6 ml to about 7 ml, about 7 ml to about 8 ml, about 8 ml to about 9 ml, about 9 ml to about 10 m, and about 3 ml to about 3.5 ml. For example the volume is about 0.25 ml to 2 ml, about 2 ml to about 4 ml, about 4 ml to about 6 ml, about 6 ml to about 8 ml, and about 8 ml to about 10 ml. In various embodiments, the effective amount of the composition is in a volume of between 100 microliters and about 5 ml, or about 5 ml to about 10 ml or about 10 ml to about 15 ml. In various embodiments, the effective amount is calculated or determined based a volume that a healthy joint holds or encompasses. For example, a healthy disc may include about 6 ml to about 10 ml, or about 10 ml to about 12 ml.

In various embodiments, identifying the mammal the having discogenic pain includes diagnosing or visualizing at least one region of the spine or the disc having the disc injury using an agent or a device. In various embodiments, the agent is at least one selected from: a contrast agent, a radiographic agent, and an imaging agent. In various embodiments, the device is at least one selected from the group of an ultrasound, a magnetic resonance imaging device, and an x-ray device. In various embodiments, the region of the spine or the disc includes a site of injury or degeneration. For example the region of the spine or the disc is found in at least one selected from: cervical, thoracic, and lumbar. For example, the disc injury involves nerve impingement or disc hernation. In various embodiments, the disc injury involves an intervertebral disc injury or condition.

In related embodiments of the method, administering the effective amount of the composition including the ELA stem cells includes contacting the composition to an implant or a graft, such that the implant or the graft includes a matrix. For example the matrix is a biocompatible material or a biocompatible polymer. For example, the biocompatible polymer includes a hydrogel biocompatible polymer selected from at least one of: 2-hydroxyethyl methacrylate and 2-hydroxypropyl methacrylate. In various embodiments the matrix includes a gel, a graft, a scaffold for example a polymer scaffold or a self-assembly scaffold, calcium triphosphate, demineralized bone, collagen, and cellulose. In various embodiments, the matrix includes collagen.

In various embodiments, administering the effective amount of the composition including the ELA stem cells includes at least one selected from the group of: a scaffold such as a collagen scaffold; a matrix; a nanocomposite for example which contains collagen-like or nucleus pulposus-like fibers, a microfiber, a nanofiber, or a nanotubule.

For example, administering the composition is performed minutes, hours, days or months after insertion of for example the implant; the graft; the nanostructure; the scaffold for example the self-assembly scaffold; the matrix; the nanocomposite; the microfiber; the nanofiber; or the nanotubule. Alternatively, administering the composition is performed minutes, hours, days or months before the insertion.

In related embodiments, the method further includes inducing the ELA stem cells to express a neurogenic factor or an osteogenic factor. Alternatively, the method further includes inducing the ELA stem cells to differentiate into a type of tissue. For example, the type of tissue is selected from: cartilage, muscle, bone, and neuronal. For example, inducing the ELA stem cells includes using at least one selected from the group of: a cell, a peptide, a protein, a drug, pressure, and energy. For example the energy includes ultrasound energy or an electrical current.

The method in various embodiments includes the step of obtaining the ELA stem cells. In various embodiments, obtaining involves collecting a population of ELA cells from the mammal, isolating the ELA stem cells and extending the ELA cells by culture prior to administering to the mammal. Alternatively, obtaining the ELA stem cells involves culturing the ELA stem cells from a stem cell bank.

In various embodiments, the method involves prior to administering culturing the ELA stem cells. In various embodiments, the culturing involves contacting the ELA stem cells with at least one of: cells, a therapeutic agent, an inorganic compound, a drug, a nucleic acid, a lipid, a protein, a peptide, an antibody, and a carbohydrate. For example, the therapeutic agent includes a drug, a nucleic acid, a lipid, a protein, a peptide for example an antibody, or a carbohydrate. For example, the cells include human cells. In various embodiments, culturing comprises using at least one from the group of: a nutrient, a buffer, a salt, a protein, a vitamin, and a growth factor. In various embodiments, the culturing promotes ELA cell growth.

In various embodiments, culturing the ELA stem cells includes contacting with nucleus pulposus. For example, culturing includes contacting the ELA stem cells with at least one of the group of: chondrocyte-like cells, a collagen, a collagen fibril, a proteoglycan, an aggrecan, and a hyaluron.

An aspect of the invention provides a method of formulating a composition for treating a condition or disorder of the spine or disc, the method including using a preparation of ELA stem cells characterized by expression of one or more stem cell-specific transcription factors, and lacking detectible expression of one or more of the cell surface markers: CD13, CD34, CD44, CD45, CD90, or MHC class I. In general, the method of formulating involves any of the compositions or formulations described herein. Without being limiting, the method further comprises a contrasting agent, an analgesic agent, or a radiographic agent. In various embodiments, the method further includes using at least one selected from the group of: a matrix, a scaffold, an implant, or graft. In various embodiments, the method involves a therapeutic agent.

### BRIEF DESCRIPTION OF THE DRAWINGS

FIG. 1 is a drawing showing an axial (overhead or vertical) view of a normal intervertebral disc having an annulus fibrosus 101 surrounding the nucleus pulposus 102. The disc is includes a T-shaped bone portion containing the spinous process (downward facing) and posterior tubercle of transverse process (left facing and right facing). The disc also includes a cavity for the spinal cord.

FIG. 2 is a drawing showing an axial view of a degenerative intervertebral disc with impingement of a nerve 201. The degenerative intervertebral disc is characterized by weakness 203 in the annulus fibrosus, 101, and prolapsed, 202 of the nucleolus pulposus 102. The prolapse 202 results in pressure on a nerve 201 in the spinal cord causing pain and other symptoms of impingement on spinal nerves.

FIG. 3 panels A, B and C are a set of drawings showing progression of a defect in an annular fibrosis.

FIG. 3 panel A is an axial (transverse view; top) drawing and a lateral (side) drawing showing an initial damage of the intervertebral disc or with a deformation of the annulus fibrosus which surrounds the nucleus pulposus.

FIG. 3 panel B is an axial drawing and an expanded axial drawing showing further deterioration of the annulus fibrosus compared to FIG. 3 panel A with nucleus pulposus extending farther posteriorly from the disc.

FIG. 3 panel C is an axial drawing and an expanded axial drawing showing severe disc herniation with complete tearing of the annulus fibrosus resulting in the nucleus pulposus flowing out of the intervertebral disc and applying pressure on the spinal cord and nerve root.

### DETAILED DESCRIPTION

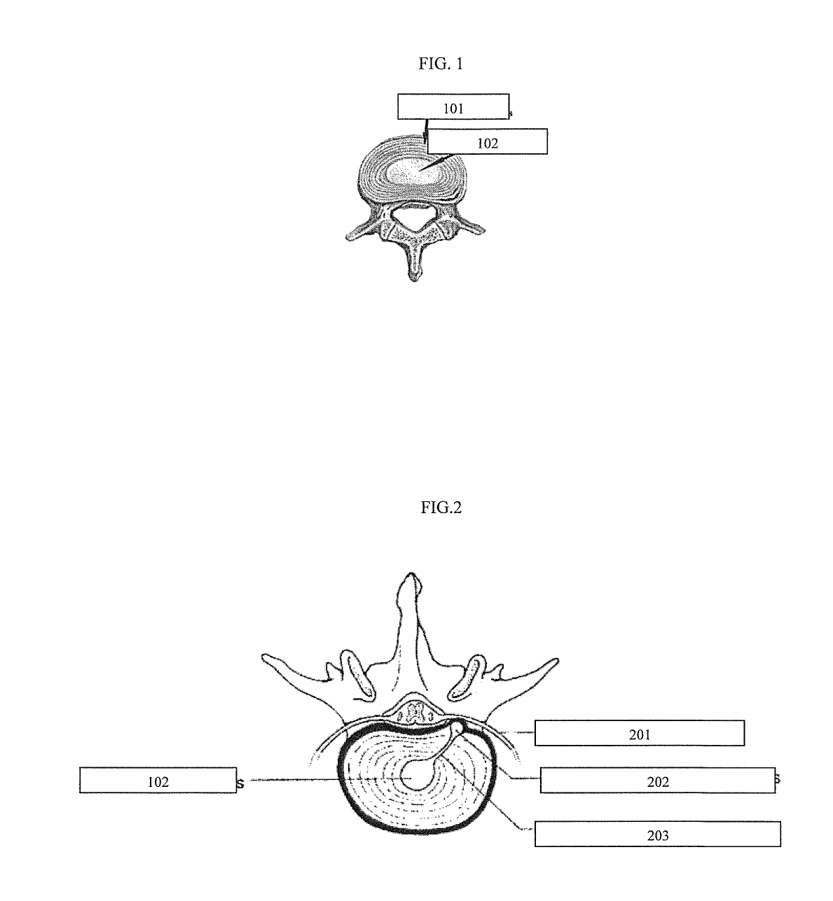

Intervertebral discs are located between adjacent vertebrae in the spine and are composed of three major structures called nucleus pulposus (FIG. 1, 102), annulus fibrosus (FIG. 1, 101), and cartilage end plates. A healthy intervertebral disc facilitates motion between adjacent vertebrae and absorbs and distributes shock forces. Injury and degeneration of the intervertebral disc occur predominantly in the cervical and lumbar spine causing compression of nerve roots and impingement on the spinal cord. Nerve root compression is associated with causation of weakness and radicular pain, generally termed radiculopathy. While radiculopathy is generally ascribed a mechanical etiology, studies have demonstrated that a chemical or inflammatory component plays a role in nerve root irritation and associated radiculopathy.

Pain often occurs without identifiable nerve compression as the degenerate or injured disc itself may be the pain generator. This has been termed “discogenic pain” and is the result of annular disruption, such as an annular tear. The various nerve endings surrounding the annulus produce pain in response to the inflammation associated with disruption of the annular rings. Often, the disruption of the annular rings is asymptomatic until the nerve roots in the outer rings are affected.

The morbidity associated with disc degeneration, disc injury and the spectrum of associated spinal disorders is responsible for significant health care, economic and social costs. Current treatment options for pain associated with disc injury and degeneration generally involve chiropractic manipulation or physical therapy, often accompanied by anti-inflammatory drugs, which are sometimes shown to have temporary pain mitigation effects but little or no success in reversing disc degeneration or repairing an injured disc. The efficacy of non-surgical therapies in repairing disc defects is limited due to the largely avascular characteristics of internal components of the disc capsule. Without the cellular and nutritional requirements needed for tissue regeneration, annular and other disc defects lack the ability to self-repair.

Alternatively, discectomy, laminectomy, laminotomy and/or spine fusion procedures are used as surgical treatments. These approaches seek neural decompression, vertebral stabilization and removal of the pain generator. Where the surgical procedure results in a discectomy (removal of the pain generator) and fusion, exacerbation of degeneration at adjacent levels may occur which require additional surgical intervention. Minimally invasive treatments, which reduce pain, reduce inflammation and reverse or moderate the deterioration of disc tissue prior to disc rupture are still needed to avoid the complications associated with disc removal. In recent experimental clinical studies, adult cells characterized as progenitor cells, i.e., having a relatively undifferentiated state, have shown some utility for certain medical applications due to their ability to facilitate tissue repair. However, to date, none of these cellular therapies have demonstrated true clinical efficacy. There remains a need in the art for cell therapies that can ameliorate discogenic pain and repair disc injury.

### ELA Stem Cells

Early lineage adult stem cells (ELA™ cells) are adult progenitor cells that have regenerative properties. ELA cells are capable of differentiating into tissues of endodermal, mesodermal and ectodermal origin. The source, isolation, characterization and certain uses and formulations of ELA cells are described in U.S. provisional application Ser. Nos. 60/927,596, 61/247,236, 61/247,242, 61/249,172, and 61/501,846, as well as U.S. application Ser. No. 12/598,047 and international PCT applications serial numbers PCT/US2008/005742 and PCT/US2010/050288, each of which is incorporated by reference herein in its entirety. Generally, the ELA cells are described by their expression of stem cell specific genes such as Oct-4, KFL-4, Nanog, Sox-2, Rex-1, GDF-3 and Stella. However, these cells are distinct from embryonic stem cells (ESC) and other types of early lineage adult cells such as mesenchymal stem cells (MSC), very small embryonic-like stem cells (VSEL), and multipotent adult progenitor cells (MAPC) and cord blood derived progenitor cells, in that the ELA stem cells do not appear to detectibly express the markers CD13, CD34, CD44, CD45, CD49a, CD66A, CD73, CD90, CD105, CSCR4, SSEA, or MHC class I or MHC class II structures.

ELA cells are may be derived from multiple sources including, bone marrow stroma, blood, dermis, periosteum and tissues, and synovial fluid (SF). The ELA stem cells in certain embodiments are utilized as fresh isolates or from cryopreserved populations including expanded populations, and are used either as autologous, syngeneic or allograft transplants, to induce the regenerative process in many types of animal or human tissues (international application number PCT/US2008/005742 filed May 5, 2008 and international application number PCT/US2010/050288 filed Sep. 24, 2010). For example, PureGen™ Cellular Allograft (available from Alphatec Spine, Carlsbad, Calif.) is a pure population ELA cells harvested from synovial fluid aspirates of live, healthy donors that are collected during routine orthopedic procedures, processed and cryopreserved into individual doses. In certain embodiments, Synovation™, a cellular matrix that comprises ELA stem cells, is used to treat a spine, a disc, a hip, an ankle, a hip, a joint, or a bone.

In various embodiments, the ELA stem cells for treating disc injuries and associated conditions are allogeneic cells or autologous cells. In various embodiments expansion of the allogeneic cells is performed using the same procedures and methods as the autologous cells. The ELA cells are for example obtained from a stem cell bank and are thawed and expanded in a suitable medium, for example a RPMI 1640 medium or a RPMI 1680 medium (Life Technologies, Invitrogen; Grand Island, N.Y.). In various embodiments the ELA cells are expanded with a medium containing serum, for example 10% fetal bovine serum. Methods for collecting ELA stem cells from bodily fluids, tissues, or stem cells banks, and expanding the ELA stem cells in media are shown in international application number PCT/US2008/005742 filed May 5, 2008 and international application number PCT/US2010/050288 filed Sep. 24, 2010, each of which is incorporated by reference herein in its entirety. But additionally herein, in various embodiments, intended to be one non-limiting example of this technology, a population of culture expanded ELA cells is formulated and used for treatment of an intervertebral disc injury, to reduce pain, and mediate inflammation resulting from the disc injury (acute or degenerative disc disease). In certain embodiments, the ELA cells are also regenerative of the disc itself. Methods using ELA stem cells described herein are effective for repairing an injured or degenerative intervertebral disc in a human patient in need of treatment.

In various embodiments, ELA cells are extracted from the patient intended to receive the cellular therapy (i.e., an autologous transplant). These ELA cells are culture expanded prior to their introduction into the patient. Growth of ELA cells in vitro is used, for example, to increase the number of ELA cells available for implantation or injection. In non-limiting examples, ELA cell numbers are increased about two-fold or greater, about ten-fold or greater, or about twenty-fold or greater. In various configurations, growing ELA cells in vitro includes expansion in a cell culture medium which comprises nutrients, buffers, salts, proteins, vitamins and/or growth factors, which promote ELA cell growth. A useful cell culture medium is for example RPMI 1680 supplemented with 10% serum, and appropriate antibiotics such as penicillin/streptomycin and G418. Human serum is used in certain embodiments for expanding the ELA cells for human transplant preparations, and fetal bovine serum has produced acceptable yields of the cells in culture. In various embodiments, the expanded cells are commonly but not necessarily frozen prior to implantation, and a suitable cryogenic medium that is acceptable to the FDA for such purposes is CryoStorCS10, available from BioLife Solutions, Bothell, Wash.

In various embodiments of the present invention, an ELA stem cell preparation is implanted or injected into an intervertebral disc of a patient in need of treatment. A preparation for injection includes in certain embodiments a high molecular weight sulfated proteoglycan composition, fibrinogen, fibrin, thrombin, type I collagen, type II collagen, type III collagen, calcium alginate, agarose, fibronectin, laminin, hyaluronic acid (HA), hydrogel, pegylated hydrogel, poly-lactic acid, poly-glycolic acid, platelet-rich fibrin, or chitosan, or combinations of these. Other agents that induce ELA cell differentiation into chondrogenic pathways are useful and are described herein. In certain aspects of these embodiments. ELA cells or tissue including ELA cells are incubated in vitro in a culture medium prior to the contacting with one or more agents described herein. Also, another useful addition to the ELA cell preparation is a perfluorocarbon agent such as perflourotributylamine, which may be combined with the above in any combinations thereof. Perfluorates increase the available oxygen to the treatment site, which is hypovascular or avascular, thereby increasing the growth and healing potential of the ELA cells. However, without limitation, exemplary exemplary effective amounts can range from 50 micrograms per milliliter to one milligram per milliliter. However, dosages below and above this range are still suitable and can be determined and titrated by the treating physician based on the specific condition of the subject or the patient.

In various embodiments ELA cell preparations are implanted or injected into an injured or degenerative intervertebral disc, i.e., into the annulus of the disc, into the nucleus pulposus of the disc, and/or one or both endplates of the disc. An aperture is formed for example in an annulus of a degenerative disc, and a composition is introduced into the disc through the aperture. Surgical techniques such as vertebroplasty and kyphoplasty (Garfin, S. R. et al. 2001 Spine 26: 1511-1515) are adapted or modified for introducing ELA cell preparations into the treatment site.

ELA cells are induced in vivo in various embodiments to express one or more characteristics of human intervertebral disc cells. In various embodiments, the ELA cell preparation is intended for repair of the annulus itself, and chondrogenic differentiation is induced by exposing the ELA cells to between about 1 to 10 micromolar insulin and between about 1 to 10 micromolar transferrin, between about 1 ng/ml and 10 ng/ml transforming growth factor (TGF) beta, and between about 10-50 nM ascorbate-2-phosphate during culture expansion. For chondrogenic differentiation, the cells are cultured in certain embodiments in high density (e.g., at about several million cells/ml or using micromass culture techniques), and also in the presence of low amounts of serum (e.g., from about 1% to about 5%). In various embodiments the ELA cell preparation is intended for repair of bone defects, osteogenic developmental phenotypes is induced by exposing the cells to between about 10-1000 nanomolar concentrations of dexamethasone in combination with about 10 nM to 50 nM ascorbate-2-phosphate and between about 10 nM to 50 nM beta-glycerophosphate. The medium in various embodiments includes serum. For amelioration of inflammation, no induction of the cells is required during culture expansion.

After culturing the cells in the differentiating-inducing medium for a suitable time (e.g., several days to a week or more), the cells in various embodiments are assayed to determine whether, in fact, they have differentiated to acquire physical qualities of a given type of cell. One measurement of differentiation per se is telomere length, undifferentiated ELA cells having longer telomeres than differentiated cells. Thus the cells in various embodiments assayed for the level of telomerase activity. Alternatively, RNA or proteins in various embodiments are extracted from the cells and assayed (QPCR, Western blot analysis, etc.) for the presence of markers indicative of the desired phenotype. Of course, the cells can be assayed immunohistochemically or stained, using tissue-specific stains. Similarly, ostogenesis in various embodiments assessed by staining the cells with bone-specific stains (e.g., alkaline phosphatase, von Kossa, etc.) or probed for the presence of bone-specific markers (e.g., osteocalcin, osteonectin, osteopontin, type I collagen, bone morphogenic proteins, cbfa, etc.). Chondrogenesis can be determined by staining the cells using cartilage-specific stains (e.g., alcian blue) or probing the cells for the expression/production of cartilage-specific molecules (e.g., sulfated glycosaminoglycans and proteoglycans (e.g., keratin, chondroitin, etc.) in the medium, type II collagen, etc.). Other methods of assessing developmental phenotype of cells are known in the art, and are appropriate for analysis of ELA cells. For example, antibodies can be employed to assess whether the ELA cells express differentiation makers.

Regardless of whether the cells are provided with one or more of the inducing agents suggested above, it is understood that the cells may be cultured or grown in an environment that has been conditioned by exposure to mature cells (or precursors thereof) of the respective type to be differentiated. Such treatment is particularly effective when the cells are provided in high density, e.g., 10⁶ cells per square centimeter to 10⁷ cells per square centimeter.

In other aspects of the present invention, to facilitate treatment and healing of a spine or a disc the ELA stem cell-containing formulation or composition is provided in a biologically compatible lattice material. In various embodiments, the lattice comprises collagen-rich material. In various embodiments, the lattice is biodegradable over a period of time (e.g., hours, days, weeks, months, and years), so that the lattice will be absorbed into the body as the ELA cell develops and replaces the damaged tissues. The lattice in various embodiments includes hormones, such as growth factors, cytokines, and morphogens (e.g., retinoic acid, aracadonic acid, etc.), desired extracellular matrix molecules (e.g., fibronectin, laminin, collagen, etc.), or other materials (e.g., DNA, viruses, other cell types, etc.) as desired. To form the ELA cell/collagen-rich lattice material, ELA cells are introduced into the lattice such that they permeate into the interstitial spaces therein. For example, the lattice can be soaked in a solution or suspension containing the cells, or the ELA stem cells are infused or injected into the lattice prior to or at the time of injection or implantation into or around the disc in need of repair. In certain embodiments, a useful formulation is a hydrogel formed by crosslinking of a suspension including the collagen-rich lattice material with the ELA cells dispersed therein. This formulation comprises ELA stem cells dispersed throughout the lattice, facilitating more even permeation of the lattice with the cells and better repair of the defect.

Lattices suitable for inclusion into the composition can be formulated de novo from many suitable sources (e.g., matrigel), and are available from commercial suppliers (e.g., suitable polyglycolic acid can be obtained from sources such as Purac Biochem., and Boehringer Ingelheim). As indicated above, a source of the collagen-rich lattice is the acellular portion of the tissue, tissue extracellular matrix matter substantially devoid of cells. In various embodiments a lattice includes proteins such as proteoglycans, glycoproteins, hyaluronins, fibronectins, collagens (type I, type II, type III, type IV, type V, type VI, etc.), and the like, which serve as excellent substrates for cell growth.

The ELA cells or the ELA cell/collagen-rich lattice material are in various embodiments combined with additional collagen-based material for use to augment a spinal disc. The additional collagen-based material is preferably derived from natural, collagen-rich tissue, such as intervertebral disc, fascia, ligament, tendon, demineralized bone matrix, etc. The material may be autogenic, allogeneic, or xenogeneic, or it may be of human-recombinant origin. Examples of collagen-rich tissues include disc annulus, fascia lata, planar fascia, anterior or posterior cruciate ligaments, patella tendon, hamstring tendons, quadriceps tendons, Achilles tendons, skins, and other connective tissues. Additionally, the lattices include hormones, cytokines or growth factors, for facilitating the growth of cells seeded into the lattice, as well as perfluorates to provide enhanced oxygenation. The ELA cell/collagen-rich lattice formulation, with or without additional collagen-based material, is provided in any form appropriate for introduction into a disc space. For example, the material in various embodiments is a solid, porous, woven, or non-woven material. The material in various embodiments is provided as a plurality of particles or small pieces, or as a fibrous material. Collagen-rich lattice material and/or the additional collagen-based material are provided in certain embodiments as particles ranging between about 0.05 millimeters (mm) and about 5 mm in size, or between about 5 mm and about 25 mm in size. When materials such as fascia lata or disc annulus particles are used as the additional collagen-based material the particles may range in size from 0.1 mm to 5 mm. When materials such as demineralized bone matrix are used the particles in various embodiments range in size from about 0.05 mm to about 3 mm, or about 3 mm to about 20 mm. The small plugs of material used the plugs in various embodiments range in size from about 0.5 mm to about 5 mm.

Cross-linking agents added in various embodiments to promote cross-linking of the collagen materials. For example, glutaraldehyde or other protein cross-linking agent is included in the formulation. The cross-linking agents promote covalent or non-covalent crosslinks between collagen molecules. Similarly, agents to inhibit protein denaturization are in various embodiments also included. When the material is to be used as a slurry or gel, additives to promote slurry or gel formation are also be included. These additives in various embodiments promote protein folding, water binding, protein-protein interactions, and water immobilization.

In addition, a radiocontrast agent, such as barium sulfate, or a radiocontrast dye, such as HYPAQUE is included in various embodiments to aid the surgeon in tracking the movement and/or location of the injected material. Other suitable radiocontrast materials appropriate for use in discography are known to persons skilled in the art. Other additives to provide surgical benefit may be included. Such additives include anesthetics, to reduce pain caused by the procedure, and antibiotics, to minimize the potential for bacterial infection.

An embodiment of the invention provides a compositions, methods, systems and kits for treating a subject having a spinal disorder or condition (e.g., lesion, fracture, and prolapse) including administering a population of ELA stem cells that do not substantially induce a T cell or natural killer (NK) cell-mediated immune response in the subject and promote tissue (e.g., bone, neural, and cartilage) ingrowth into a defect site associated with the spinal disorder. In various embodiments, administering the ELA stem cells involves administering an implant including ELA stem cells or administering ELA stem cells during or after inserting an implant or a graft, such that the implant or the graft is described herein. Methods for preparing and administering ELA stem cells in implants, scaffolds, or grafts are shown for example in Crawford et al. international application number PCT/US2010/050288 filed Sep. 24, 2010, which is incorporated by reference in its entirety. In various embodiments, the ELA stem cells are administered to implants, grafts, or scaffolds to enhance healing of the disc disorder or condition. For example, ELA stem cells are administered minutes, hours, days or months after an implant, graft, or scaffold is inserted. Alternatively, administering the ELA stem cell-containing composition or formulation is performed minutes, hours, days or months before an implant, graft, or scaffold is inserted.

Implants and bone grafts used in current orthopedic and neurosurgery procedures often serve different functions. Methods for preparing and administering stem cells with implants or grafts are shown in Fernandes et al. international application number PCT/US2007080428 filed Oct. 4, 2007; Barry et al. international application number PCT/US2001/013267 filed Apr. 24, 2001; and Edinger et al. international application number PCT/US2007/022545 filed Oct. 23, 2007. In certain situations, a graft is needed to act as a scaffold to aid the new bone in completely bridging a defect. In certain embodiments of the graft, the material is porous to allow for bone ingrowth into the graft. Porous bone grafts act as a scaffold or trellis that allows regenerating bone to heal across a defect that it normally is not otherwise treatable (Borden U.S. Pat. No. 8,110,007 issued Feb. 7, 2012). Cancellous bone and porous ceramics are used in various embodiments as bone graft scaffolds. For example three-dimensional microsphere scaffolds are made by coating a porous microsphere scaffold with a thermally stable material (Borden et al. U.S. Pat. No. 8,177,854 issued May 15, 2012). The scaffold is porous for bone and cartilage to grow through the material and into the site of injury.

In certain embodiments, the implant or graft includes a disc stabilization device for example an intradiscal stabilization device. In one embodiment, the intradiscal implant includes a hydrogel or a microgel. For example, the implant is a pH-responsive microgel particle effective to repair an injury. The implant undergoes a conformational change in response to the pH in the body.

In various embodiments, the ELA stem cells are administered with a self-assembly scaffold or self-assembly matrix. In various embodiments, the scaffold or matrix is biocompatible. For example the self-assembly scaffold or matrix is liquid at room temperature and upon injection into the body or a body cavity changes into a solid form or semi-solid (e.g. a gelatinous or collagen-like consistency) form.

The composition including the ELA stem cells is administered prior to, contemporaneously, or subsequent to a matrix, a scaffold, an implant, or graft. For example, the ELA stem cells are administered with a biocompatible matrix (e.g., a temperature dependent self-assembly, nanotubules, or nanofibers) into the disc place or any cavity with a targeted-access cannula, equipped for example with a fluid entry port. The cannula in various embodiments are part of a disposable and sterile kits, which allow the injection of the ELA cell-biocompatible matrix into disc, knee, shoulder, elbow, and ankle spaces. In various embodiments, administering the ELA stem cells and either a matrix, a scaffold, an implant, or graft in performed in an office without the need for bio-fluid pumping, anesthesia or sterilization support infrastructures.

The implant, graft or device in various embodiments is treated with ELA stem cells to enhance healing of the disc condition or disorder, or to reduce pain, inflammation and discomfort in the subject. (Hunt et al. U.S. patent publication number 2008/0045949 published Feb. 21, 2008; and Sanders et al. U.S. publication number 2008/0254133 published Oct. 16, 2008). In another embodiment, the intradiscal implant is an artificial nucleus pulposus which supports or replaces the existing nucleus pulposus, or a portion thereof, of the intervertebral disc. In an additional embodiment, the artificial nucleus pulposus contains a load bearing polymer. e.g., a polyethylene and a polyester (Jin et al. U.S. patent publication number 2012/0010599 published Jan. 12, 2012). In still another embodiment, the artificial nucleus pulposus contains a non-load bearing polymer. In a further embodiment, the artificial nucleus pulposus comprises a biocompatible hydrogel. In still another embodiment, the artificial nucleus pulposus includes a biomaterial selected from the group consisting of collagen type I, chytosan, fibrin, alginate, hyaluronate, cellulose, glycolide (PGA), polylactide (PLA) foam, and polyacrilonitril.

In various embodiments, structural implants or grafts are constructed as bone graft materials whose main function is to mechanically support the site and add stability during the healing process. In various embodiments the materials have little to no porosity and have the strength necessary for stabilization (Burton, U.S. patent publication 20050096652 published May 5, 2005). Cortical bone struts, rings, and wedges are examples of structural grafts. For example methods for stabilizing intervertebral joints involve attaching a stabilization device composed of anchors that are attached to the joints by screws or fasteners. In certain embodiments, the implant or graft is a load-bearing device that stabilizes the disc or spine. For example, the load-bearing device with the ELA stem cells is inserted into a posterior region of the vertebral column (U.S. publication numbers: 20070213718 published Sep. 13, 2006; 20070213822 filed Sep. 13, 2007; 2007/0213823 filed Sep. 13, 2007; 2007/0213824 filed Sep. 13, 2007; 2007/0227547 published Oct. 4, 2007, each of which is incorporated by reference in its entirety). In certain embodiments, the materials are natural (e.g., bone) or synthetic (e.g., plastics and polymers). For example, the natural materials are further treated to have the desired characteristics, e.g., porosity, hardness, and strength.

The source of the bone-derived grafts is in various embodiments either from the patient's own cancellous or cortical bone (autograft) or from a tissue donor, i.e., an allograft (Semler international application number PCT/US2007/016528 filed Jul. 23, 2004). Although these bone grafts have been used to treat defect, however they have certain disadvantages. Removing healthy bone from the patient and placing at another site often results in complications of pain and infection at the donor site. Using a tissue donor can result in variable resorption characteristics and unpredictable structural integrity. In an attempt to avoid the problems of autograft and allograft, synthetics have become a popular choice for orthopedic surgeons. In various embodiments, the intervertebral disc treatment devices are fibrous plugs inserted into the cavity of the disc. The device strengthens the annulus of the disc and prevents escape of the nucleus pulposus through the annulus (McKay et al. U.S. Pat. No. 7,318,840 issued Jan. 15, 2008). Resorbable polymers, ceramics, and composites are in various embodiments effective substitutes in certain embodiments for bone derived grafts without any of the autograft or allograft related complications.

Porous implants are used in various embodiments as graft materials for bone and cartilage repair and are used in various embodiments as scaffolds. These scaffolds have in various embodiments a high percent porosity to allow for bone and/or cartilage in-growth. A variety of pore forming techniques to create three-dimensional porous scaffolds are used, e.g., drilling and punching, in various embodiments. These techniques, however, sometimes result in structures that may be easily crushed or deformed due to their low strength. In orthopedic grafting procedures, surgeons often use force to impact the graft material into the site, which may crush or fracture the graft material. Thus, if the graft material has low mechanical properties, the porosity is significantly reduced or completely eliminated if the graft is crushed or fractured during or after the surgical procedure. With little to no porosity remaining, the graft no longer functions as a scaffold for tissue in-growth. In various embodiments, the tissue scaffolds used herein are constructed and prepared using materials that they have sufficient strength to prevent crushing during the implantation procedure.

For structural grafting applications, implants in various embodiments composed of non-degradable materials such as titanium, poly(ether ether ketone) which is referred to as PEEK, and a polyethylene, a poly(orthoester) (POE), a poly(L-lactic acid) (PLA), or a polysulfone (PSF). In various embodiments, high strength materials are used that possess the required mechanical properties, and which do not negatively affect the healing process. Once the site has fully healed, the implant may no longer serve an useful purpose and can be a source of long term complications such as loosening or failure. In various embodiments, resorbable implants are constructed and used that perform their mechanical function and are then resorbed by the body. Polymers such as poly(lactic acid) (PLA) and poly(lactide-co-glycolide) (PLG) are used in various embodiments to create resorbable implants for structural applications such fracture fixation, reconstruction, and spinal fusion. Alternatively a composition composed of fibrous polypeptide (e.g., spider silk and elastin) attached to a polysaccharide binding domain, e.g., a chitin binding domain or a hyaluronan binding domain, is used in certain embodiments that enhances the strength and elasticity of the implant (Shoseyov et al. international application number PCT/IL2008/01542 filed Nov. 26, 2008).

Typically resorbable polymer implants maintain their volume until the very end of resorption. This can lead to a void in the tissue that may or may not eventually fill in. In certain embodiments, sterile non-fusion implants composed of cancellous bone are constructed that are compressable and upon hydration expands to the pre-compression form (Gertzman et al. U.S. patent publication number 2006/0235534 published Oct. 19, 2006). In various embodiments, insertable implants are constructed that are composed of demineralized, non-osteoinductive cancellous bone tissue in a compressible mesh material. The bone and mesh in certain embodiments are compressed for insertion into the annulus area of the spine, and strengthen the nucleus pulposus for distributing hydraulic pressure in all directions within each disc under compressive load (Semler et al. U.S. Pat. No. 7,959,683 issued Jun. 14, 2011). Bioactive molecules (i.e., peptides, proteins, genetic material, and carbohydrates) are applied and embedded in grafts and implants to enhance the integration, stability, and therapeutic effective of the grafts and implants.

The benefits of the inventive materials and methods described herein include augmentation of the intervertebral disc and restoration and improvement of the natural condition and/or performance of the disc and or reduce inflammation or pain. In addition, augmentation can retard or reverse the progressive degeneration of an injured or diseased disc. Particular formulations and processes of the present invention are not limited to the descriptions of the specific embodiments presented, but rather the descriptions and examples should be viewed in terms of the claims that follow and their equivalents.

ELA stem cells described herein for application to spinal and disc defects are treated in various examples herein with various other types of therapeutic agents or stimuli to promote tissue formation, healing, pain relief, and inflammation reduction. Different techniques are used to enhance healing of joints and tissues using ELA stem cells, e.g., cells, peptides, proteins, drugs, and electrical current.

ELA stem cells for treatment of spinal defects and disc defects are treated in various examples herein with various other types of stimuli to promote tissue formation and healing. Platelets secrete a number of factors including serotonin, fibronectin, ADP, thromboxane A, platelet factor 4, platelet-derived growth factor, and platelet activating factor that are effective for spinal and disc repair (Long et al. U.S. Pat. No. 8,343,480 issued Jan. 1, 2013). Release of these factors causes a chemotactic response which initiates the process of migration between endothelial cells. As more factors continue to be released from platelets as well as monocytes and macrophages, angiogenesis, osteogenesis and the formation of granulation tissue are promoted. Several platelet-based products are commercialized under the labeling of Platelet Rich Plasma (PRP), also referred to as Autologous Platelet Gel. The commercial products include the UPS® System (Biomet), Fibrinet (Cascade Medical), and SmartPrep (Harvest), which are described for use following arthroscopic repairs, such rotator cuff repair, meniscus repair, and ACL reconstruction. These PRP systems are disclosed as containing several growth factors, including vascular endothelial growth factor, platelet-derived growth factor, transforming growth factor-P, fibroblast growth factor and epidermal growth factor. Fibroblast growth factor-2 enhances functional recovery of innervated muscle, e.g., following surgery (Iwata et al. 2006 Muscle & Nerve 34(5):623-630).

Autologous plasma rich in growth factors are used in certain embodiments herein to restore connective tissues following arthroscopic treatment of large, non-traumatic avulsions of articular cartilage in the spine, disc or knee (Sanchez et al. 2003 Med. Sci. Sports Exerc. 35(10):1648-1652. Foster, et al., Am. J. Sports Med. 37(11):2259-72 (2009), is a review that reports on the use of platelet-rich plasma for chronic tendinopathy, bone healing, acute ligamentous injuries, total knee arthroplasty, ACL reconstruction, acute Achilles tendon repair, rotator cuff repair, acute cartilage and meniscus repair. Intervertebral disc treatment devices in various embodiments are constructed with a compressible fibrous body and an effective amount of a tissue growth factor and additional cells or therapeutic agents. The body of the device in various embodiments is compressible to pass through an opening in the annulus fibrosus and expandable to prevent expulsion through the opening (McKay et al. U.S. patent publication 2002/0173851 published Nov. 21, 2002 and McKay et al. U.S. patent publication number 20060004456 published Jan. 5, 2006). A sterile non-fusion implant composed of cancellous bone that is compressable is constructed that upon hydration expands to the pre-compression form (Gertzman et al. U.S. patent publication number 2006/0235534 published Oct. 19, 2006). Bioactive molecules or cells (e.g., plasmids, hormones, growth factors, stromal cells and mesenchymal stem cells) are added to the implant to improve function of the implant (Ibid.). Alternatively, a central nervous system disorder or pain is further treated by administering a TNF inhibitor such as Rituximab (Shafer et al. U.S. patent publication 2005/0095246 published May 5, 2005).

In certain embodiments, the ELA stem cells are administered with a therapeutic agent that binds to DNA or modulates transcription. In certain embodiments, the therapeutic agent is a transcription factor. For example, the stem cells are administered to reduced pain in the body (e.g., spine and disc) using for example a nuclear factor kappa-light-chain-enhancer of activated B cells (Burright et al. international application number PCT/US2007/071593 filed Jun. 19, 2007).

In certain embodiments, the ELA stem cells are induced with osteoinductive agents, for example the ELA stem cells are treated with the bone morphogenetic proteins or BMPs, or are treated to express BMPs. When coupled with an osteoconductive carrier, BMPs are used in certain embodiments for treatment of bone conditions (Valentin-Opran, A. et al. 2002 Clin Orthop, 395: 110-120). A spine disc prosthesis device is converted for example into a fusion device by using bone forming material agent such as a BMP (Johnson U.S. patent publication number 2012/0136447 published May 31, 2012). Alternatively, bone growth is induced in stem cells by transfecting the cells with a vector encoding a LIM mineralization protein. The vector induces BMP expression in the cell (McKay international application number PCT/US2004/007616 filed Mar. 7, 2004). Upregulated gene expression of the BMP super family (e.g., BMP-2, BMP-44, and FGF-2), alkaline phosphatase, and osteocalcin is achieved in various embodiments in endogenous stem cells by applying specific and selective signals to coils, electrodes, or other field generating devices adjacent the ELA stem cells, bone or adjacent tissue cells (Brighton U.S. Pat. No. 8,313,908 issued Nov. 20, 2012; and Brighton U.S. patent publication number 2012/0149968 published Jun. 14, 2012).

ELA stem cells are treated in certain embodiments by administering a peptide or peptide-encoding RNA, for example a NELL peptide (a protein strongly expressed in neural tissue encoding epidermal growth factor like domain) or a NELL-encoding RNA, that induces bone formation (see Soo et al. international application number PCT/US2010/002297 filed Aug. 20, 2010; Soo et al. U.S. patent publication number 2011/0300184 published Dec. 8, 2011; Soo et al. international application number PCT/US2006/005473 filed Feb. 16, 2006; and Soo et al. U.S. patent publication number 2009/0053311 published Feb. 26, 2009). For example a composition comprising ELA stem cells are contacted with a stem cell binding peptide. The stem cell binding peptide, which is identified for example by an affinity selection technique such as biopanning, induces bone growth and repair (Melican et al. U.S. patent publication number 2011/0117171 published May 19, 2011; and Melican et al. international application number PCT/US2010/057266 filed Nov. 18, 2010). In another attempt to treat an annular defect, a biocompatible material is inserted into a spinal defect such that the material plugs the defect. Additional cells (e.g., chondrocytes, and bone marrow cells) or therapeutic agents (e.g., growth factors) in various embodiments are added to the biocompatible material or to a composition including the ELA stem cells to enhance disc treatment.

In certain embodiments, the ELA stem cells are treated with growth factors, osteoblasts, chondroblasts, mesenchymal stem cells, and fibroblast (Lin et al. international application number PCT/US2005/039938 filed Nov. 3, 2005; and (McDonald U.S. patent publication number 2009/0081273 published Mar. 26, 2009)). In certain embodiments, the amelioratory effects of ELA stem cells is enhanced by electrical current or electric fields. For example the ELA stem cells are electrically stimulated in the subject's spinal column (Grandjean et al. U.S. patent publication number 2009/0117088 published May 7, 2009).

Without being limited by any particular theory or mechanism of action, it is envisioned that formulations including ELA cells express specific proteins or molecules that are therapeutic to the spinal defect or disc defect of a subject. Delivery of the ELA cells is performed through various modes including topical or direct application (as for example by solutions, ointments, or drops), injection into or through dermal, epithelial, muscle and subcutaneous sites, or using a device. See McLachlin et al. 1990 Progress in Nucleic Acid Research and Molecular Biology 38: 91-135; Gerson et al. U.S. Pat. No. 5,591,625 issued Jan. 7, 1997; and Schroff, U.S. publication number 2009/0123433 published May 14, 2009). Spinal or disc injections include direct injection into the intervertebral disc, or injection into adjacent or external layers of the spinal tissue (e.g., epidural space and facet joint), such as injection into at least of the cervical region, thoracic region, or lumbar regions of the spine. Methods that are well known to those skilled in the art are used to construct expression vectors containing a sequence encoding a peptide or a protein operably linked to appropriate transcriptional and translational control elements. These methods include in vitro recombinant DNA techniques, synthetic techniques and in vivo recombination or genetic recombination. Such techniques are described in Sambrook et al., Molecular Cloning: A Laboratory Manual, Cold Spring Harbor Press, Plainview, N.Y., 1989. For example the vector is at least one selected from an adenovirus, an adeno-associated virus, a herpesvirus, a poxvirus, and a lentivirus.

For example, the ELA stem cells are administered using a device (e.g., a catheter device). For example, the device includes a reservoir for delivery of stem cells to the disc (e.g., annular pulposus and nucleus pulposus) of a subject. See Walsh U.S. patent publication number 2011/0040279 published Feb. 17, 2011.

The ELA cell/collagen-based material is “surgically added” or injected to the disc space. That is, the material is added by the intervention of medical personnel, as distinguished from being “added” by the body's natural growth or regeneration processes. The surgical procedure preferably includes injection through a hypodermic needle or surgical implantation, although other surgical methods of introducing the collagen-based material into the disc may be used. For example, the material may be introduced into a disc by extrusion through a dilated annular opening, infusion through a catheter, insertion through an opening created by trauma or surgical incision, or by other means of invasive or minimally invasive deposition of the materials into the disc space. In various embodiments, the ELA stem cells are administered using an imaging system. For example, the ELA stem cells are administered in the doctor's office or during a doctor's visit. In various embodiments, the ELA stem cells are administered using an arthroscopic imaging system designed specifically for in-office diagnostic testing. For example, the imaging system includes a endoscope such as a VisionScope Imaging system that utilizes a 1.4 mm diameter endoscope, providing physicians the ability to visualize, assess and definitively diagnose joint pathologies during the office visit.

Without being limited by any particular theory or mechanism of action, it is envisioned that vectors are used to transfect ELA cells to express specific proteins or molecules that are therapeutic to the spinal or disc defect and to the recipient subject. Delivery of the transformed cells is effected through various modes including topical or direct application or injection into dermal, epithelial, muscle and subcutaneous sites. See McLachlin et al. 1990 Progress in Nucleic Acid Research and Molecular Biology 38: 91-135; and Gerson et al. U.S. Pat. No. 5,591,625 issued Jan. 7, 1997, each of which is incorporated by reference herein in its entirety.

### Pharmaceutical Compositions or Formulations

An aspect of the present invention provides pharmaceutical compositions or formulation that includes a population of ELA stem cells for treating a spine or disc-related disorder or condition, e.g., disc herniation. The ELA stem cells induce tissue growth, and reduce pain, discomfort, and inflammation.

In related embodiments, the pharmaceutical composition is formulated sufficiently pure for administration to a human subject or a portion thereof, e.g., to the spinal column and an invertebral disc. In certain embodiments, these compositions optionally further include one or more additional therapeutic agents. In certain embodiments, the additional therapeutic agent or agents are selected from the group consisting of growth factors, anti-inflammatory agents, vasopressor agents including but not limited to nitric oxide and calcium channel blockers, collagenase inhibitors, topical steroids, matrix metalloproteinase inhibitors, ascorbates, angiotensin H, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGFs), IGF binding proteins (IGFBPs), epidermal growth factor (EGF), platelet derived growth factor (PDGF), neu differentiation factor (NDF), hepatocyte growth factor (HGF), vascular endothelial growth factor (VEGF), heparin-binding EGF (HBEGF), thrombospondins, von Willebrand Factor-C, heparin and heparin sulfates, and hyaluronic acid.

In certain embodiments, a plurality of therapeutic agents are include in the pharmaceutical composition to treat the same, a concurrent or a related symptom, condition or disease. In some embodiments, the therapeutic agent is a drug that may include without limitation anti-coagulant, anti-tumor, anti-viral, anti-bacterial, anti-mycobacterial, anti-fungal, anti-proliferative or anti-apoptotic agents. Drugs that are included in the compositions of the invention are well known in the art. See for example, Goodman & Gilman's The Pharmacological Basis of Therapeutics, 9th Ed., Hardman, et al., eds., McGraw-Hill, 1996, the contents of which are herein incorporated by reference herein in their entireties.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 provides various carriers used in formulating pharmaceutical compositions and known techniques for the preparation thereof. Some examples of materials which can serve as pharmaceutically acceptable carriers include, but are not limited to, sugars such as glucose and sucrose; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, preservatives and antioxidants can also be present in the composition, the choice of agents and non-irritating concentrations to be determined according to the judgment of the formulator.

### Therapeutically Effective Dose

Methods provided herein involve contacting cells or tissues in a subject with a pharmaceutical composition or formulation containing a therapeutically effective amount of ELA stem cells. The ELA stem cells are administered in such amounts and for such time as is necessary to achieve the desired result including reduction or preventing of indicia of a spine or disc-related disorder or condition.

The compositions, according to the method of the present invention, may be administered using any amount and any route of administration effective for treating the spine or disc-related disorder or condition. Thus, the expression “amount effective for treating a spine or disc-related disorder or condition”, as used herein, refers to a sufficient amount of composition to beneficially prevent or ameliorate the symptoms of the disease or condition affecting the spine or the disc. For example the disease or condition is a scoliosis, degenerative disc disease, kyphosis, cervical spondylosis, failed back surgery syndrome (FBSS), herniated nucleus pulposus, slipped disc, lumbar radiculopathy, cervical radiculopathy, herniated intervertebral disc, prolapsed intervertebral disc, ruptured disc, spinal surgery, disc surgery, or ankylosing spondylitis. Other joints such as knees, ankles, wrists are administered different suitable volumes appropriate for that joint. Without being limited, the volume administered to joints is about 0.25 microliters to about 50 milliliters.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., intermediate or advanced stage of disc herniation or disc degeneration; age, weight and gender of the patient; diet, time and frequency of administration; route of administration; drug combinations; reaction sensitivities; level of pain, and tolerance/response to therapy. Long acting pharmaceutical compositions might be administered hourly, twice hourly, every three to four hours, daily, twice daily, every three to four days, every week, once every two weeks, monthly, bi-monthly, every six-months, or yearly depending on the condition of the disc and the specific circumstances of the subject or patient.

The active agents of the invention are preferably formulated in dosage unit form for ease of administration and uniformity of dosage. The expression “dosage unit form” as used herein refers to a physically discrete unit of active agent (i.e., ELA stem cells) appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays, in vitro models, or in animal models, as provided herein, usually mice, but also potentially from rats, rabbits, dogs, or pigs. The animal cell model and in vivo model described herein are also used to achieve a desirable concentration and total dosing range and route of administration. Such information can then be used to determine useful doses and routes for administration in humans.

A therapeutically effective dose refers to that amount of active agent that ameliorates the symptoms or condition or prevents progression of the disease or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and animal studies are used in formulating a range of dosage for human use.

The daily dosage of the products may be varied over a wide range, such as from 100 ELA cells to 10¹¹ ELA stem cells. For example, the therapeutic dose per disc per subject may be at least about 10² cells to about 10³ cells, about 10³ cells to about 10⁴ cells, about 10⁴ cells to about 10⁵ cells, about 10⁵ cells to about 10⁶ cells bout 10⁶ cells to about 10⁷ cells; 10⁷ to about 10⁸ cells; about 10⁸ cells to about 10⁹ cells; about 10⁹ cells to about 10¹⁰ cells; and about 10¹⁰ cells to about 10¹¹ cells. As ELA stem cells are readily obtained (as described herein) and are inexpensively cultured and stored, greater doses for administration are economically feasible. For animals several orders of magnitude smaller or larger than humans, or for smaller or larger humans than those used in examples herein, the dose is easily adjusted, for example, to about 500 or 3×10² ELA stem cells, or about 3×10¹¹, to 3×10¹² ELA stem cells or about 3×10¹³ ELA stem cells. Doses of about 3×10¹⁴, 3×10¹⁵ ELA stem cells, or even about 3×10¹⁶, or about 3×10¹⁷ ELA stem cells, for example are within the scope of the invention. For preventive treatments, or periodic treatment, or treatment of, smaller doses such as less than about 100, 500, 100, 3×10², or less than about 3×10³, or even less than about 3×10⁴ per dose, are within the scope of the invention.

Administration of a source of expression of a therapeutic agent or modulator (e.g., a peptide or a protein) to treat ELA stem cells is within the scope of the invention. In certain embodiments, the therapeutic agent or the modulator enhances differentiation or function of the ELA stem cells, for example the therapeutic agent is a peptide or a protein that induces differentiation, e.g., osteogenic. Administration of a dose of a viral vector or a nucleic acid vector that encodes a therapeutic agent or a modulator, for example the dose contains at least about 50, 100, 500, 1000, or at least about 5000 particles per cell to be treated. Alternatively, the dose of a viral vector or a nucleic acid vector is at least about 10⁴ to about 10⁵; about 10⁵ to about 10⁶; 10⁶ to about 10⁷; 10⁷ to about 10⁸; about 10⁸ to about 10⁹; about 10⁹ to about 10¹⁰; or at least about 10¹⁰ to about 10¹¹. The dose effective for treating a cell number can be calculated from the number of ELA stem cells or the area in need of treatment by methods known to one of skill in the art.

### Administration of Pharmaceutical Compositions or Formulations

As formulated with an appropriate pharmaceutically acceptable carrier in a desired dosage, the pharmaceutical composition provided herein is administered to humans and other mammals for example topically (as by powders, ointments, or drops), subcutaneously, subdermally, submusclarly, or parenterally depending on treatment objectives and the severity and nature of a spine or disc-related disorder or condition.

Injections include inter-discal injection or intra-discal injection in the spine of the subject, or injection into adjacent tissues or external layers of the disc, for example by injection outside of the annulus pulposus or within an epidural space.

Liquid dosage forms for example for inter-discal or intra-discal, or other administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the ocular, oral, or other systemically-delivered compositions can also include adjuvants such as wetting agents, and emulsifying and suspending agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, discal routes of administration are achieved with aqueous drops, a mist, an emulsion, or a cream. Administration may be therapeutic or it may be prophylactic. The invention includes discal devices, implant devices, spine devices or products which contain disclosed compositions (e.g., gauze bandages or strips), and methods of making or using such devices or products. These devices may be coated with, impregnated with, bonded to or otherwise treated with a composition as described herein.

Transdermal patches containing therapeutic agents for enhancing the ELA stem cells have the added advantage of providing controlled delivery of the therapeutic ingredients to the spine and body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Solid dosage forms for topical administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

The following examples and claims are illustrative only and not intended to be further limiting. Those skilled in the art will recognize or be able to ascertain using no more than routine experimentation, numerous equivalents to the specific procedures described herein. Such equivalents are within the scope of the present invention and claims. The contents of all references including issued patents and published patent applications cited in this application are hereby incorporated by reference.

Reference will now be made to specific examples using the processes described above. It is to be understood that the examples are provided to more completely describe particular embodiments, and that no limitation to the scope of the invention is intended thereby. All publications, patents, patent applications and other references cited in this application are herein incorporated by reference in their entirety as if each individual publication, issued patent, patent application or other reference were specifically and individually indicated to be incorporated by reference.

### Examples

### Example 1

### ELA Stem Cell Treatment of a Patient with Discogenic Pain

A 53-year old male patient having discogenic pain resulting from an L2 annular tear is presented for treatment. The nerve roots in the outer rings are affected. The various nerve endings surrounding the annulus show minor damage from responses to inflammation associated with disruption of the annulus, resulting in chronic pain surrounding the injury site. Pain is quantitatively assessed using a subjective self-assessment, based on common pain-intensity scales. The scales range from 0 for little or no pain, 1-3 for mild pain, 4-6 for moderate pain, 7-10 for severe pain and 10 being the worst pain the patient has ever experienced. The patient complains of chronic pain levels between 5 and 7 during daytime hours. The patient also suffers from osteoarthritis of the knee, and aspiration of synovial fluid incident with treatment of that disorder has resulted in collection of the SF. The patient has elected to expand these cells for treatment of the unrelated disc condition.

### Example 2

### A Culture Expanded Autograft Formulation of ELA Cells and Treatment with Same

SF is centrifuged gently to pellet the cells, and the cells are resuspended in Hybri-Max RBC lysing buffer (Sigma-Aldrich, St. Louis, Mo.) to lyse enucleated cells (red blood cells) while keeping the nucleated cells (ELA stem cells) intact. The cells are collected by centrifugation and washed in a sterile isotonic buffer solution prior to enrichment. The enriched ELA cells are characterized by expression of stem cell specific genes such as Oct-4, KFL-4, Nanog, Sox-2, Rex-1, GDF-3 and Stella, and lack of detectible expression of one or more of the markers CD13, CD34, CD44, CD45, CD49a, CD66A, CD73, CD90, CD105, CSCR4, SSEA, MHC class I or MHC class II. Enrichment to 98% or greater is desirable. The enriched ELA cells number approximately 10² from the collection procedure, and these are culture expanded prior to formulation. Approximately 10̂7 ELA cells are obtained from culture extension, and these are cryopreserved for shipping to the clinic in connection with surgical treatment of the injury site.

In the clinical setting, the physician prepares the final formulation, which in this example includes collagen, a hydrogel matrix, radiographic contrast dye, perflourotributylamine, an analgesic, and saline. Preferably, these are supplied with the cells in the form of a single use kit. These are added to the ELA cells until the final volume is about 0.75 milliliters and the cell density is approximately 1.25 million cells per milliliter. Injection is accomplished with a 24 gauge needle, and the ELA cell formulation is injected directly into the nuclear disc space through the annulus. The final composition is contained within the disc space following injection as verified by radiography. Excess fluid including the radiographic contrast dye and saline subsequently diffuse out of the disc space and leave the cells and final matrix behind.

For repair of the annulus a single injection is desirable. Subsequent to radiography, a second injection of a different ELA cell formulation is delivered to the extraspinal space, to ameliorate inflammation surrounding the nerve roots and subsequent pain therefrom. In this example, the formulation includes antibiotics, an analgesic, cortisone, and saline to a final volume of 2.0 milliliters. The ELA cell concentration is approximately 2.5 million cells per milliliter.

Following treatment (two days to seven days) the patient is assessed for clinical improvement as determined by a reduction in self-reported pain. Radiographic assessment of the annulus after about 30 days will show improvement in structure.

### Example 3

### Collection and Purification of ELA Stem Cells

ELA stem cells are obtained from blood of mammalian subjects which is harvested, diluted in serum-free medium (GIBCO™ AIM V Medium liquid; Invitrogen, Carlsbad, Calif.), and centrifuged three times at 200 g for five minutes to ten minutes at room temperature. The cells are then re-suspended in serum-free medium, and then are layered over a combined gradient of Ficoll-Paque and Stem Cells Technologies Granulocyte gradient commercially available from ROSETTE SEP DM-M; Stem Cell Technologies, Vancouver, British Columbia. Granulocytes are prevented by the gradient and red blood cells and stem cells are obtained in to pellet. The sample is centrifuged at 500 g for 30 minutes at room temperature. The Ficoll-Stem Cell Technology gradients separate the cells into a bully layer, an intermediate layer, and pelleted layer.

The desired ELA stem cell population is isolated from the pelleted layer, washed in phosphate-buffered saline, and resuspended in culture medium. See U.S. utility application Ser. No. 12/598,047 filed Oct. 29, 2009, publication US 2010/0291042 published Nov. 18, 2010.

### Example 4

### Culture and Analysis of ELA Stem Cells

ELA stem cells are inoculated in culture vessels (5,000 cells/cm² per vessel) containing culture medium, Dulbecco's Modified Essential Medium (DMEM) with low glucose (DMEM-LG) and chick fibroblast basal medium (MCDB 201) containing insulin-transferrin-selenium (ITS), linoleic acid-bovine serum albumin (LA+BSA), dextrose, L-ascorbic acid, platelet-derived growth factor (PDGF), epidermal growth factor (EGF), insulin-growth factor (IGF-1), penicillin/streptomycin, and 2% fetal bovine serum (FBS). Cells are grown to confluence and multiple expansion cultures are also obtained. See PCT/US2010/50288 filed Sep. 24, 2010

The ELA stem cells are analyzed for expression of adult stem cell markers using quantitative polymerase chain reaction (QPCR) analysis. The data show that the ELA cells do not express MHC class I, MHC class II, CD44, CD45, CD13, CD34, CD49c, CD73, CD105, and CD90 cell surface markers. These cell surface markers to induce or are associated with an immune response in subjects, thus the ELA stem cells herein are not immunogenic stem cells.

### Example 5

### Injection of ELA Stem Cells after Administering a Disc Implant

Disc implants are inserted and then subjects administered ELA stem cells to determine whether the ELA stem cells enhanced healing post-disc implant compared to subjects administered only disc implants.

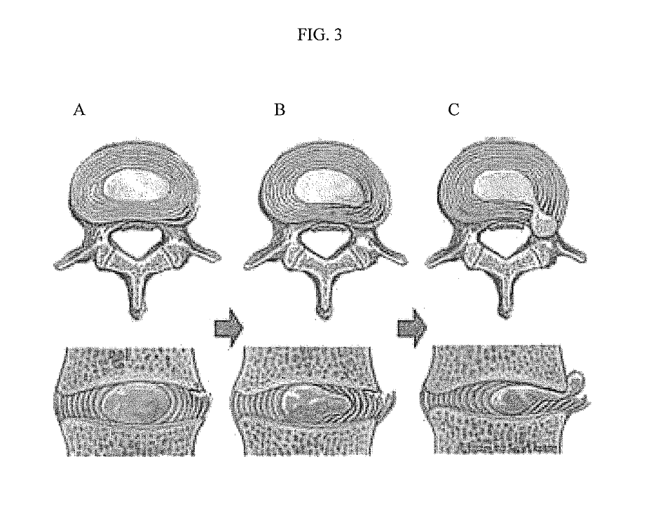

Demineralized disc implants are prepared and are inserted into the site of herniation of the annulus fibrosus 101. A drawing of an exemplary intervertebral disc and an herniated intervertebral disc is in FIG. 1 and FIG. 2 respectively. The weakness 203 and deterioration of the annulus fibrosus 101 over time allows the soft, central portion (i.e., the nucleus pulposus 102) to bulge out beyond the damaged outer rings to produce a prolapse 202 that places pressure on the nerve 201. This deterioration and eventual tear in the disc ring may result in the release of inflammatory chemical mediators which may directly cause severe pain, and compression of the nerve. (FIG. 3 panels A-C).

The device is inserted into the disc space such that the device plugs the defect in the annulus fibrosus. The device is situated by fasteners or screws in the defect such that it presses against the inner annulus fibrosus to prevent the device from be shifting and allowing the nucleolus pulposus to prolapse.

Minutes, hours or days post-disc implant insertion, the area of the disc is injected with ELA stem cell compositions. The final ELA stem cells composition includes different amounts and concentrations of components such as collagen, a hydrogel matrix, radiographic contrast dye, perflourotributylamine, an analgesic, and saline. The components are added to the ELA cells until the final volume is about 0.5 milliliters to 1.5 milliliters, and the cell density is approximately 500,000 to 2.5 million cells per milliliter.

Data show that the ELA stem cells irrespective of the cell density enhance healing and repair of the site of the disc injury compared to control subjects administered the disc-implant only. Further the ELA stem cells reduce inflammation, pain, and discomfort compared to subjects administered the disc implant only.

1. A formulation comprising: a preparation of ELA stem cells characterized by expression of one or more stem cell-specific transcription factors, and lacking detectible expression of one or more of the cell surface markers: CD13, CD34, CD44, CD45, CD90, and MHC class I.

2. The formulation according to claim 1 further comprising at least one selected from: a pharmaceutically acceptable excipient, a contrast agent, an analgesic agent, a radiographic agent, a buffer, a cryogenic agent, and a therapeutic agent.

3. (canceled)

4. The formulation according to claim 2, wherein the contrast agent comprises a perfluorate agent.

5. (canceled)

6. (canceled)

7. The formulation according to claim 1, further comprising at least one selected from the group of: a matrix, a scaffold, an implant, a cadaveric tissue, and a graft and wherein at least one of the matrix, the scaffold, the implant, and the graft is biocompatible.

8. (canceled)

9. The formulation according to claim 7, wherein the scaffold is a self-assembly scaffold that is liquid at about room temperature and becomes a solid, a semi-solid, or a gel within a subject.

10. The formulation according to claim 1, wherein the preparation of ELA stem cells is culture expanded and contains cultured ELA stem cells.

11. The formulation according to claim 1, wherein the ELA stem cells are obtained from at least one selected from: a stem cell bank, and a cadaveric donor.

12. The formulation according to claim 1, wherein the ELA stem cells are autologous or allogenic.

13. (canceled)

14. The formulation according to claim 10, wherein the cultured ELA stem cells are differentiated.

15. (canceled)

16. The formulation according to claim 2, wherein the therapeutic agent includes at least one selected from: an inorganic compound, a drug, a nucleic acid, a lipid, a peptide, a protein, and a carbohydrate.

17. The formulation according to claim 16, wherein the nucleic acid comprises a DNA, a cDNA or an RNA selected from: mRNA, tRNA, rRNA, siRNA, RNAi, miRNA, and dsRNA.

18. (canceled)

19. (canceled)

20. The formulation according to claim 2, wherein the therapeutic agent includes a vector or a plasmid, wherein the vector carries a nucleotide sequence that encodes a peptide or a protein.

21. (canceled)

22. The formulation according to claim 16, wherein the protein is a growth factor.

23. The formulation according to claim 2, wherein the therapeutic agent is at least one selected from: a fibroblast growth factor (FGF) for example FGF2; an endothelial growth factor (EGF); a stem cell factor (SCF); an interleukin (IL) for example IL-6 or IL-2; a transforming growth factor (TGF) for example TGF-α or TGF-β; and a combination there of.

24. The formulation according to claim 2, wherein the therapeutic agent is a human peptide or a human protein.

25. The formulation according to claim 1, wherein the composition is effective for treating at least one selected from: a knee, a shoulder, a hip, an ankle, and a wrist, and is effective for reducing at least one selected from: pain, discomfort, and inflammation.

26. (canceled)

27. A method for treating disc injury in a mammal comprising:
identifying a mammal having discogenic pain incident to an annular lesion or a degenerative disc;
administering an effective amount of a formulation of ELA stem cells and within or in association with the annulus or the degenerative disc, wherein the ELA stem cells are characterized by expression of one or more stem cell-specific transcription factors, and lacking detectible expression of one or more of the cell surface markers: CD13, CD34, CD44, CD45, CD90, and MHC class I, thereby treating the annular lesion or degenerative disc, and thereby ameliorating or reducing pain in the mammal.

28. The method according to claim 27 wherein the effective amount of the formulation comprises a population of the ELA stem cells at a cell density between 10⁴ and 10⁷ cells per milliliter.

29. The method according to claim 27, the effective amount of the formulation comprises a volume of between 0.25 milliliters and 10 milliliters.

30. The method according to claim 27 further comprising prior to administering to the mammal the step of obtaining from the mammal a population of the ELA stem cells, isolating the ELA stem cells and extending the ELA stem cells by culture.

31. The method according to claim 27, wherein administering comprises contacting adjacent tissues with the composition and allowing migration or diffusion of the ELA stem cells to the annulus or the degenerative disc.

32. The method according to claim 27, wherein administering involves topical administration or injection.

33. (canceled)

34. The method according to claim 32, wherein injecting comprises at least one selected from the group of: intraspinally, spinally, epidurally, intravenously, and intra-muscularly.

35. The method according to claim 27, wherein administering involves using a cannula, a needle, a syringe, or an endoscope.

36. The method according to claim 27, wherein identifying the mammal the having the discogenic pain includes diagnosing or visualizing at least one region of the spine or the disc having the disc injury using an agent or a device.

37. The method according to 36, wherein diagnosing or visualizing comprises using at least one selected from the group of: a contrast agent, a radiographic agent, and an imaging agent.

38. The method according to claim 27, wherein administering the effective amount of the composition including the ELA stem cells further includes contacting with at least one selected from the group of: a scaffold; a matrix; a nanocomposite, a microfiber, a nanofiber, and a nanotubule.

39. The method according to claim 38, wherein the scaffold is the self-assembly scaffold which is in liquid form at room temperature, and administering the scaffold in the mammal results in the scaffold solidifying or becoming semi-solid.

40. The method according to claim 27 further comprising prior to administering, culturing the ELA stem cells.

41. The method according to claim 40, wherein culturing involves contacting the ELA stem cells with at least one of: at least one cell, a therapeutic agent, an inorganic compound, a drug, a nucleic acid, a lipid, a protein, a peptide, an antibody, a carbohydrate a nucleus pulposus, a chondrocyte-like cells, a collagen a collagen fibril, a proteoglycan, an aggrecan, and a hyaluron.

42. (canceled)

43. (canceled)

44. A kit for treating disc injury in a mammal comprising:
a formulation containing a preparation of ELA stem cells characterized by expression of one or more stem cell-specific transcription factors, and lacking detectible expression of one or more of the cell surface markers: CD13, CD34, CD44, CD45, CD90, and MHC class I;
instructions for use; and,
a container.

45. The kit according to claim 44 further comprising at least one selected from the group of: a matrix, a growth factor, an analgesic, a radiographic agent, a therapeutic agent, and an agent that modulates expression of a gene or gene product in ELA stem cells.

46. (canceled)

47. (canceled)

48. A method of formulating a composition for treating a condition or disorder of the spine or disc, the method comprising using a preparation of ELA stem cells characterized by expression of one or more stem cell-specific transcription factors, and lacking detectible expression of one or more of the cell surface markers: CD13, CD34, CD44, CD45, CD90, and MHC class I.

49. The method according to claim 48, further comprising at least one selected from: a contrast agent, an analgesic agent, and a radiographic agent.

50. The method according to claim 49, wherein the contrast agent comprises a perfluorate agent.

51. (canceled)

52. (canceled)

53. The method according to claim 48, further comprising at least one selected from the group of: a matrix, a scaffold, an implant, and graft.

54. (canceled)
